(19) United States
(12) Patent Application Publication
(10) Pub. No.: US 2016/0324986 A1
(43) Pub. Date: Nov. 10, 2016

(54) COMPOSITIONS AND METHODS FOR PROVIDING ACTIVE TELOMERASE TO CELLS IN VIVO

(71) Applicant: TELOREGEN, INC., Orion, MI (US)

(72) Inventors: Douglas COYLE, Aurora (CA); Michael FOSSEL, Ada, MI (US)

(21) Appl. No.: 15/108,013

(22) PCT Filed: Dec. 22, 2014
(86) PCT No.: PCT/US14/71991

§ 371 Date: Jun. 24, 2016

Related U.S. Application Data
(60) Provisional application No. 61/921,235, filed on Dec. 27, 2013.

(51) Int. Cl. A61K 48/00; A61K 38/45; A61K 9/127

(52) U.S. Cl. CPC A61K 48/0008; A61K 9/1271; A61K 48/005; A61K 48/0066; A61K 38/45; A61K 48/0075; C12Y 207/07049

(57) ABSTRACT

This invention provides liposomes for delivering to target cells in a subject, nucleic acids for expressing telomerase reverse transcriptase and/or telomerase RNA component. Expression of active telomerase can extend the length of telomeres in the cell. Such lengthening can be useful in subjects suffering from diseases associated with shortened telomeres.

### CROSS-REFERENCE TO RELATED APPLICATIONS

This application claims the benefit of the filing date of US. Provisional application 61/921,235, filed Dec. 27, 2013.

### STATEMENT AS TO FEDERALLY SPONSORED RESEARCH

None.

### BACKGROUND OF THE INVENTION

1. Field of the Invention

Described here is the systemic or targeted delivery of gene medicines for the extension of telomeres to living cells in an organism. More particularly described are the combination of liposome technology, pegylation technology, receptor-mediated transcytosis technology, receptor-mediated endocytosis technology, nuclear signaling endosome technology, nuclear membrane translocation technology, therapeutic gene technology, and plasmid technology to provide formulations that are useful in the systemic or targeted delivery of gene medicines for the extension of telomeres to living cells throughout an organism.

2. Description of Related Art

Over the past few decades, researchers have come to understand that the progression of human aging is governed at the cellular level by the shortening of structures called telomeres at the tips of chromosomes in each of an organism's cells. As telomeres shorten with repeated cell divisions, gene expression changes within cells, resulting in a progressive slow down and eventual halting of the process of repair of daily wear and tear, and in the progressive malfunction of cells, tissues and the entire organism. Shortening of telomeres is a component of aging and age-related disorders.

Studies indicate that re-extension of telomeres by telomerase can restore aspects of youthful functioning of cells, tissues and organisms. (Ronald A. DePinho, Richard Saltus, Dana-Faber Cancer Institute, “Partial reversal of aging achieved in mice”, Harvard Gazette, Nov. 28, 2010.)

Providing such a telomere extension or reextension therapy has proven challenging to date. Small molecule activators of the endogenous telomerase gene have proven, so far, to be only marginally effective.

It is difficult to safely deliver effective quantities of large molecules such as RNA, DNA and proteins to a significant number of cells throughout the body. Capillary walls, the blood-brain barrier, cell membranes and the nuclear membrane all present formidable barriers to large molecules and the blood, plasma and cytosol all contain many defense or immune mechanisms intended to destroy such foreign invaders.

Recombinant proteins, such as recombinant versions of the Telomerase Reverse Transcriptase (TERT) component of telomerase, tend not to fold properly, when expressed exogenously, and generally do not result in the formation of competent telomerase. It has proven difficult to achieve a sufficient cellular concentration of telomerase by introducing copies of the TERT protein.

Viral vectors have proven to be of low effectiveness.

### SUMMARY OF THE INVENTION

In one aspect provided herein is a method of expressing telomerase in a target cell in a subject comprising administering to the subject a pharmaceutical composition comprising a liposome and a pharmaceutically acceptable carrier; wherein the liposome comprises a PEG-ylated lipid membrane having an external surface and defining an internal compartment, wherein: a) the external surface has attached thereto a targeting agent directed against a receptor on a target cell involved in receptor-mediated endocytosis or macropinocytosis; and b) the internal compartment contains a nucleic acid vector comprising an expression control sequence operative in the target cell and operatively linked to a nucleotide sequence encoding telomerase reverse transcriptase (TERT); wherein the liposome is endocytosed or macropenocytosed by the target cell, and the target cell expresses active telomerase. In one embodiment the internal compartment contains nucleic acid vector comprising an expression control sequence operative in the target cell and operatively linked to a nucleotide sequence encoding telomerase RNA component (TERC). In another embodiment the subject is a vertebrate, e.g., a mammal, e.g. a human. In another embodiment the pharmaceutical composition is administered intravenously, intrathecally, intra-articularly, intraocularly, intramuscularly, orally, parenterally or topically. In another embodiment the pharmaceutical composition is administered intravenously, and wherein the external surface of the liposome has attached thereto a targeting agent directed against a receptor on an endothelial cell of an endothelial barrier involved in receptor-mediated transcytosis, wherein the liposome crosses the endothelial barrier. In another embodiment the endothelial barrier is the blood brain barrier. In another embodiment the targeting agent directed against the receptor on the target cell is also directed against the receptor on the endothelial cell. In another embodiment the target cell is a cell of the nervous system and the endothelial cell is a cell of the blood brain barrier. In another embodiment the target cell is a cell of the cardiovascular system; the digestive system; the endocrine system; the urinary system; the immune system; the musculoskeletal system; the nervous system; the reproductive system; or the respiratory system. In another embodiment the liposome is administered intra-articularly and wherein the liposome comprises a targeting agent for a receptor on a chondrocyte. In another embodiment the subject suffers from a disease associated with shortened telomeres. In another embodiment the active telomerase extends the length of shortened telomeres in the subject.

In another aspect provided herein is a method of treating a subject suffering from a disease associated with shortened telomeres comprising administering to the subject a liposome, wherein the liposome comprises a PEG-ylated lipid membrane having an external surface and defining an internal compartment, wherein: a) the external surface has attached thereto a targeting agent directed against a receptor on a target cell involved in receptor-mediated endocytosis or macropinocytosis; and b) the internal compartment contains a nucleic acid vector comprising an expression control sequence operative in the target cell and operatively linked to a nucleotide sequence encoding telomerase reverse transcriptase (TERT); wherein administration results in expression of active telomerase in target cells of the subject and wherein the active telomerase extends the length of telomeres in the target cells. In one embodiment expression of telomerase is transient (e.g., expression for up to any of 4 hours, 8 hours, 24 hours, 2 days, 4 days, or 8 days). In another embodiment the administration produces between 100 copies and 100,000 copies of telomerase in the target cell. In another embodiment the disease associated with shortened telomeres is selected from Alzheimer's disease, arterial sclerosis, osteoporosis and progeria. In another embodiment the disease associated with shortened telomeres is selected from a condition in Table I. In another embodiment the method comprises administering a liposome to the subject a plurality of times.

In another aspect provided herein is a liposome for delivering at least one nucleic acid vector encoding telomerase reverse transcriptase to a target cell, wherein the liposome comprises a PEG-ylated lipid membrane having an external surface and defining an internal compartment, wherein: a) the external surface has attached thereto a targeting agent directed against a receptor on a target cell involved in receptor-mediated endocytosis or macropinocytosis; and b) the internal compartment contains a nucleic acid vector comprising an expression control sequence operative in the target cell and operatively linked to a nucleotide sequence encoding telomerase reverse transcriptase (TERT). In one embodiment the target cell is a cell of the nervous system, e.g., a neuron (e.g., a pyramidal cell, a Purkinje cell, a granule cell or another resident neuron), a glial cell (e.g., a microglial cell, an astroglial cell, an oligodendrocyte or another resident glial cell), or a transient cell entering the nervous system. In another embodiment the target cell is a cell of the cardiovascular system; the digestive system; the endocrine system; the urinary system; the immune system; the musculoskeletal system; the nervous system; the reproductive system; or the respiratory system. In another embodiment the targeting agent is directed toward an insulin receptor. In another embodiment the targeting agent is directed toward a target other than one that mediates formation of endosomes destined for cell destination other than the nucleus, e.g., other than transferrin receptor for low-density lipid receptor. In another embodiment the targeting agent comprises monoclonal antibody directed against the receptor. In another embodiment the targeting agent is selected from an endogenous peptide ligand of the receptor, an analog of the endogenous peptide ligand, or a monoclonal antibody that binds the receptor. In another embodiment the targeting agent is selected from insulin, insulin-like growth factor (IGF), and leptin. In another embodiment the external surface has attached thereto a targeting agent directed against a receptor on an endothelial cell of an endothelial barrier involved in receptor-mediated transcytosis. In another embodiment the endothelial barrier is the blood brain barrier. In another embodiment the targeting agent directed against a receptor on the target cell is also directed against the receptor on the endothelial cell. In another embodiment the target cell is a cell of the nervous system and the endothelial cell is a cell of the blood brain barrier. In another embodiment at least one nucleic acid vector comprises a plasmid. In another embodiment the TERT is a human TERT or a modified version thereof. In another embodiment the nucleotide sequence encoding TERT comprises a naturally occurring nucleotide sequence encoding human TERT. In another embodiment the expression control sequence comprises an SV40 promoter. In another embodiment at least one expression control sequence comprises a promoter specific for the target cell. In another embodiment the internal compartment further contains nucleic acid vector comprising an expression control sequence operative in the target cell and operatively linked to a nucleotide sequence encoding telomerase RNA component (TERC). In another embodiment TERC is human TERC. In another embodiment the nucleotide sequence encoding TERT and the nucleotide sequence encoding TERC are comprised in the same vector. In another embodiment the nucleotide sequence encoding TERT and the nucleotide sequence encoding TERC are operatively linked to the same expression control sequence. In another embodiment the internal compartment contains nucleic acid vector comprising an expression control sequence operative in the target cell and operatively linked to a nucleotide sequence encoding a therapeutic biomolecule other than telomerase reverse transcriptase or telomerase RNA component. In another embodiment the therapeutic biomolecule is LMNA A, e.g., human LMNA A. In another embodiment the therapeutic biomolecule is Elongation Factor 1 (ELF1).

In another aspect provided herein is a pharmaceutical composition comprising a liposome of this disclosure and a pharmaceutically acceptable carrier. In one embodiment the pharmaceutical composition is formulated for intravenous, intrathecal, intra-articular, intraocular, intramuscular, oral, parenteral or topical administration.

In another aspect provided herein is a unit dosage form comprising a container containing a pharmaceutical composition comprising a liposome of this disclosure and a pharmaceutically acceptable carrier.

### BRIEF DESCRIPTION OF THE DRAWINGS

FIG. 1 depicts intravenous administration of a liposome bearing a targeting agent against a target on the blood brain barrier and a target on a glial cell. The liposome crosses the blood brain barrier (BBB) by transcytosis and deposits its contents into the glial cell by endocytosis.

FIG. 2 depicts lengthening of telomeres by recombinant telomerase delivered with a targeting liposome. A liposome containing a plasmid and bearing a targeting agent binds to a receptor on the target cell. Contents of the liposome are taken into the cell through endocytosis, and the plasmid translocates to the nucleus. The plasmid includes a promoter (Pr) operatively linked to a nucleotide sequence encoding TERT. The TERT sequence is transcribed, producing a TERT mRNA. The TERT mRNA is translated into telomerase reverse transcriptase protein. Telomerase reverse transcriptase protein associates with telomerase RNA component to produce active telomerase. In the nucleus, telomerase lengthens telomeres of chromosomes.

FIG. 3 depicts a targeting liposome of this invention. The liposome includes a hydrophobic bilayer. The liposome surface is derivatized with PEG. Certain PEG moieties are coupled with targeting agents. In this example a first targeting agent is directed toward a receptor involved in receptor-mediated transcytosis and a second targeting agent is directed to a receptor involved in receptor-mediated endocytosis. The hydrophilic core of the liposome contains a plasmid including a promoter (Pr) operably linked with a nucleotide sequence encoding telomerase reverse transcriptase and, in this example, a second promoter operably linked with the nucleotide sequence enclosed encoding telomerase RNA component.

FIG. 4A-B shows a nucleotide sequence for a cDNA for human telomerase reverse transcriptase (TERT). (SEQ ID NO:1.) (See also U.S. Pat. No. 6,337,200 (Morin).)

FIG. 5 shows an amino acid sequence for human telomerase reverse transcriptase. (SEQ ID NO:2.) (See also US patent U.S. Pat. No. 6,337,200 (Morin).)

FIG. 6 shows a genomic nucleotide sequence for telomerase RNA component. (SEQ ID NO:3.) (See also U.S. Pat. No. 6,013,468 (Andrews et al.).)

FIG. 7 shows a nucleotide sequence for telomerase RNA component (TERC). (SEQ ID NO:4.) (See also U.S. Pat. No. 6,013,468 (Andrews et al.).)

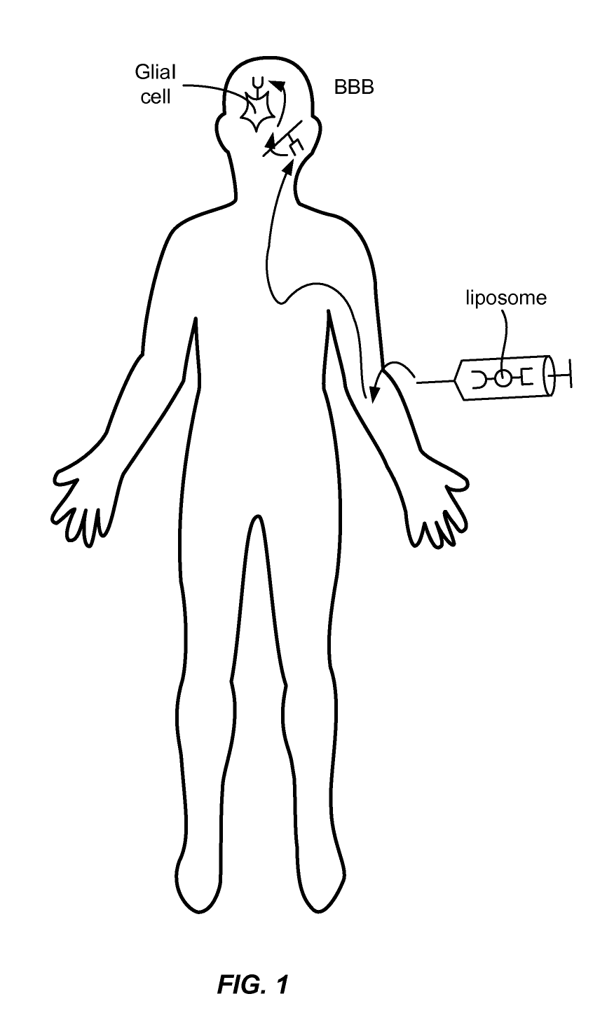

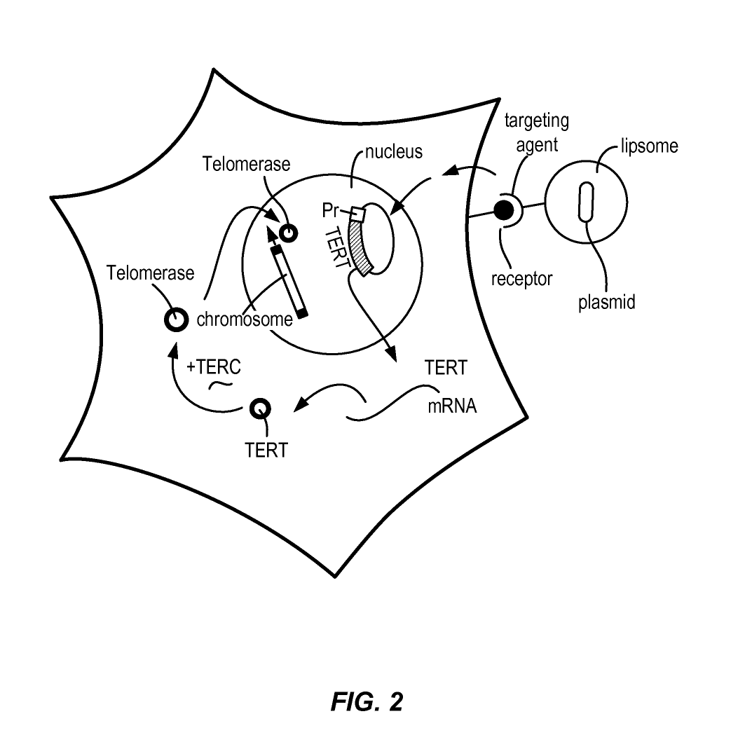

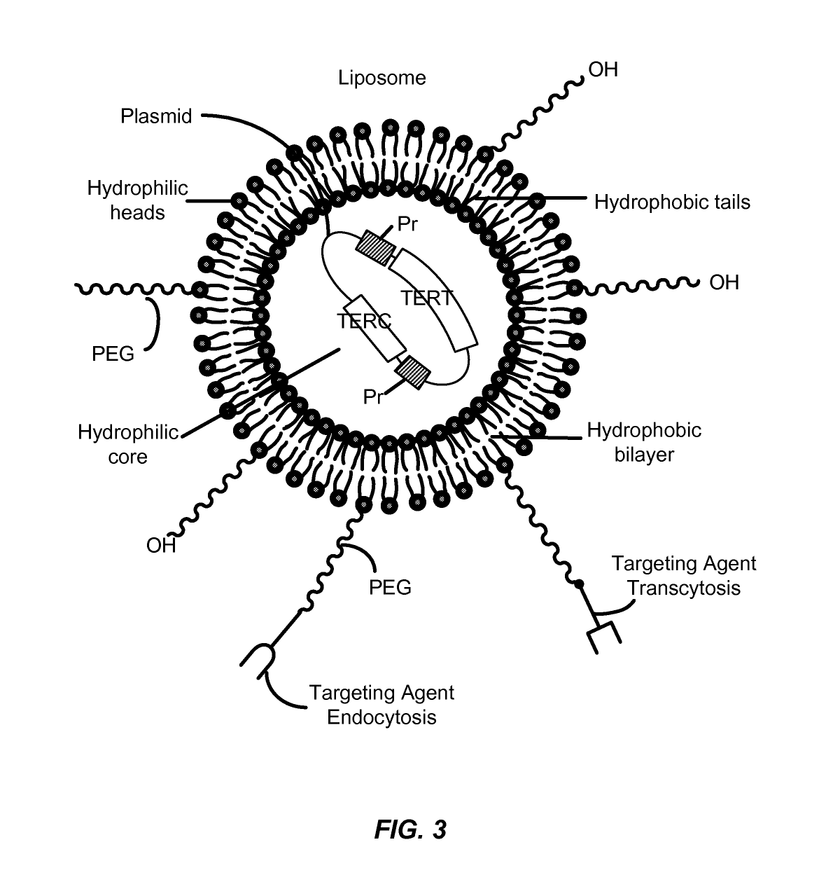

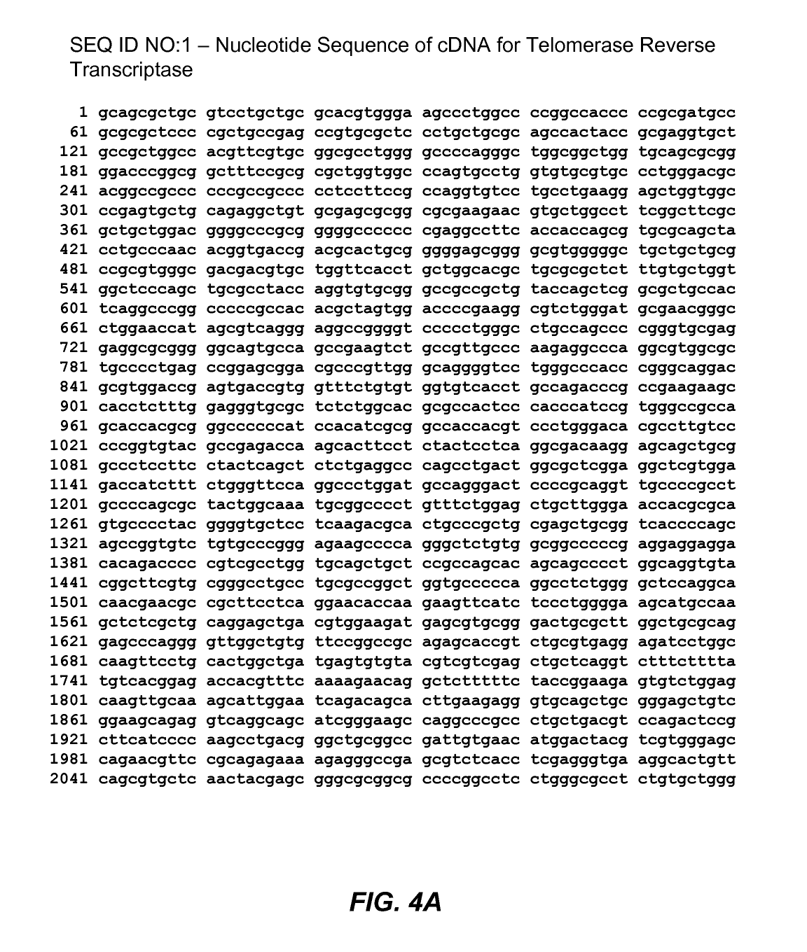

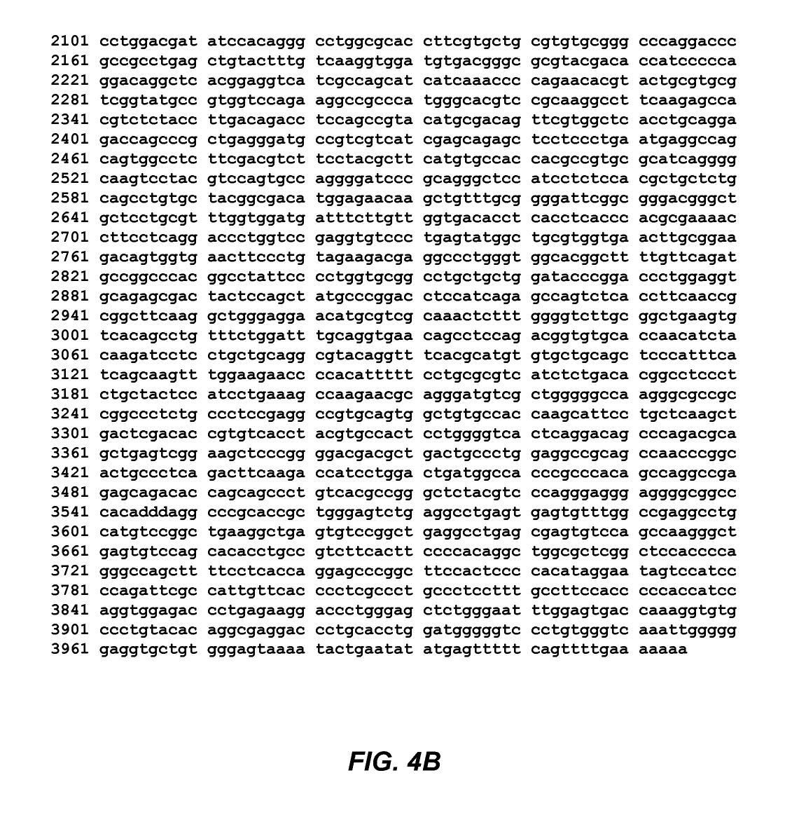

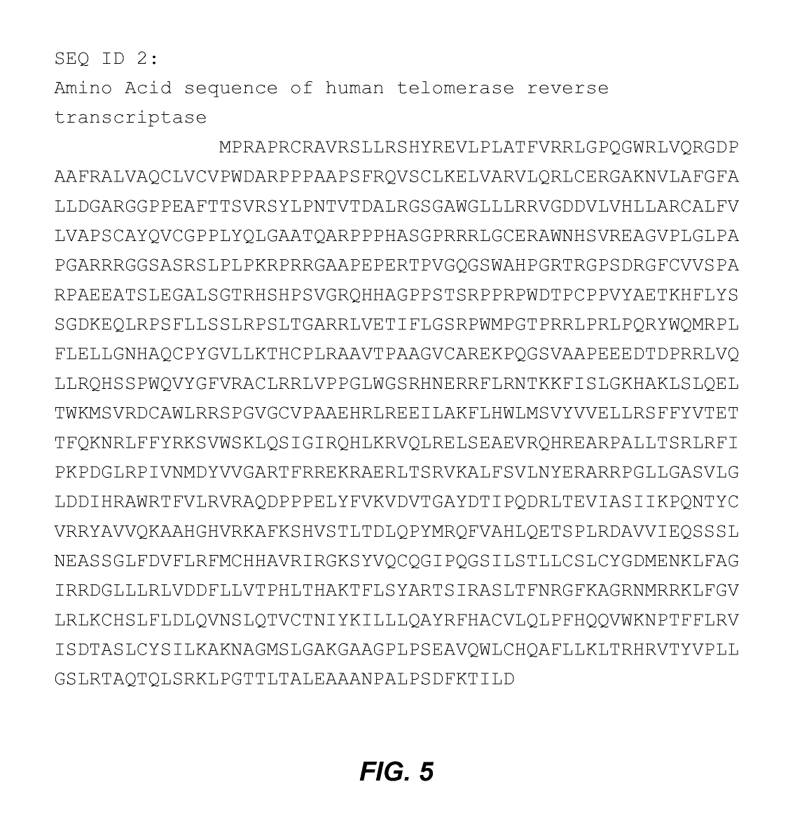

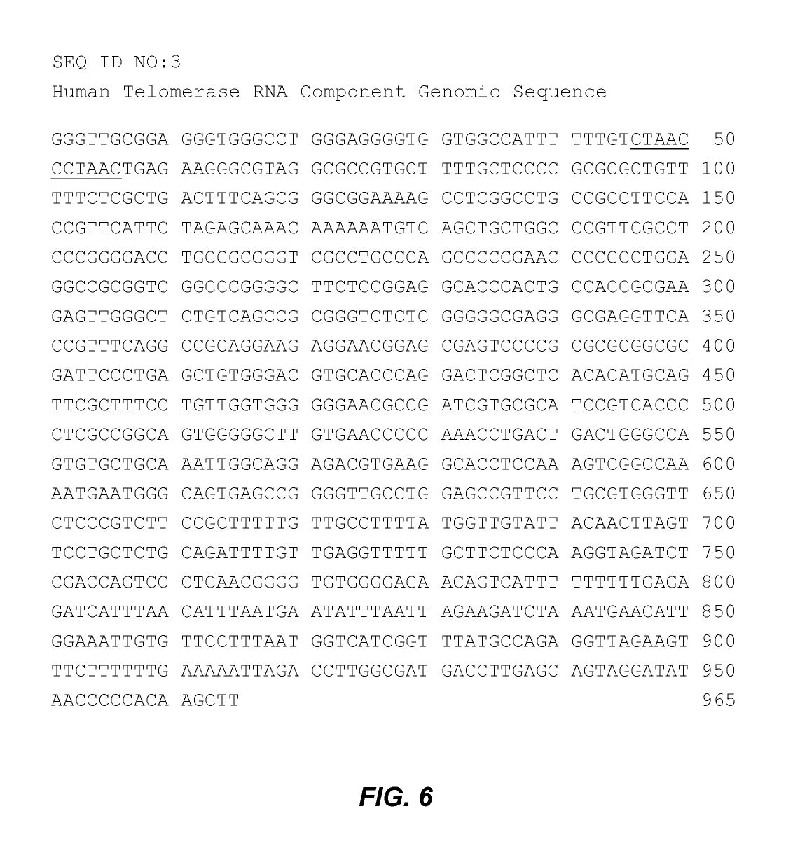

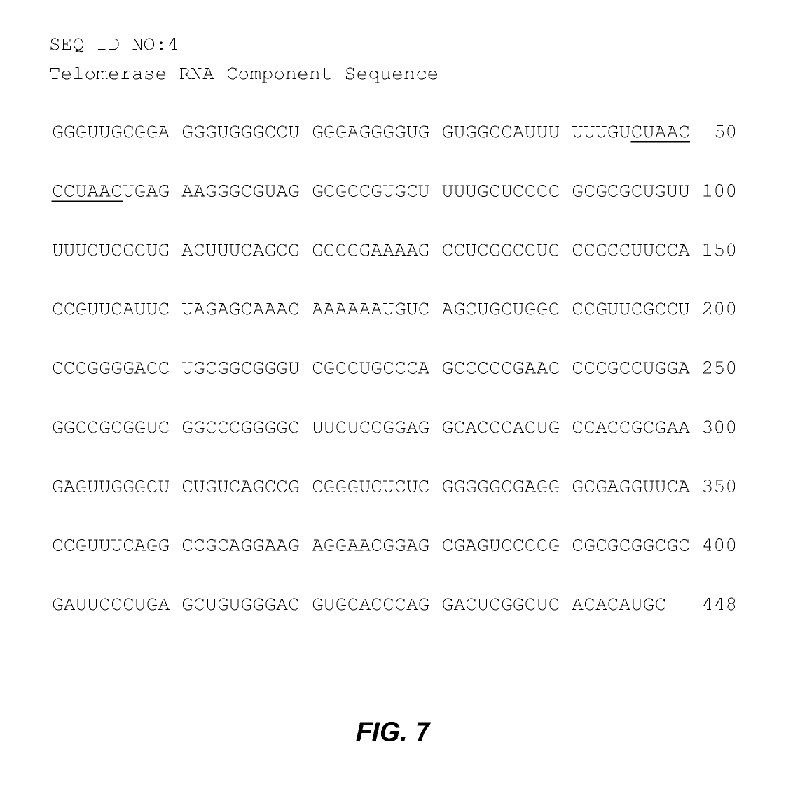

### DETAILED DESCRIPTION OF THE INVENTION

Provided herein are methods to deliver to target cells throughout an organism an exogenous gene-based therapy that can result in the transient native expression of a high cellular concentration (at least 100 copies per cell) of competent telomerase over a sufficient period of time, e.g., 4 hours, or 24 hours or 48 hours, that would re-extend chromosomal telomeres by thousands of base pairs, rejuvenating the cells, after which the expression of telomerase would return to normal levels for the cell type, and normal cell aging would resume as cell divisions occur, acting as a defense against cancers. Such a therapy can safely be repeated for a subject periodically as required, indefinitely.

This invention provides compositions and methods for delivering a nucleic acid molecule encoding telomerase reverse transcriptase (“TERT”) and, optionally, telomerase RNA component (“TERC”) to a target cell. In the cell, the nucleic acid molecule directs expression (transcription and translation) of telomerase reverse transcriptase protein. TERT associates with TERC to produce active telomerase. Telomerase functions in the cell to extend the telomeres of chromosomes. The length of telomeres in chromosomes in a cell is positively correlated with the lifespan, the functionality and the proliferative capacity of the cell.

A composition of this invention for delivering a nucleic acid molecule encoding TERT to a target cell includes a targetable liposome, sometimes referred to as a “Trojan horse liposome”. Liposomes comprise a membrane having an exterior surface and defining an interior compartment. Liposomes of this invention include one or more targeting agents on their surface and one or more nucleic acid molecules having a nucleotide sequence encoding TERT in the interior compartment. Targeting agents are selected that bind to receptors on target cells. Binding effects endocytosis of the liposome and its contents into a cell, translocation of a delivered nucleic acid to the cell nucleus and expression of nucleic acids under the control of promoters active in the cell.

Where the targeting agent also is directed to a moiety on the endothelial barrier, or, alternatively, where the liposome is provided with a different targeting agent directed to such a moiety, the liposome can undergo transcytosis across the endothelial barrier. Accordingly, to achieve targeted or systemic delivery of the liposome it is not necessary to deliver a liposome directly to the compartment that contains the target cell. Rather, it suffices to administer the liposome to a location where it can gain access to the compartment by crossing the endothelial barrier. For example, a liposome can carry a targeting agent that binds to a receptor on a cell of a capillary wall. Such a liposome, administered intravenously, can cross the capillary by transcytosis and, thereby, gain access to the compartment in which the target cell resides. So, for example, if the target cell is in the brain, a liposome that comprises a targeting agent against the receptor on both the blood brain barrier and brain cell can be administered intravenously and deliver its contents to the brain cell.

Expression of telomerase in the target cell lengthens telomeres in the cell. Accordingly, in one aspect, this invention provides methods of lengthening telomeres in cells of a subject. Such subjects can include those suffering from diseases characterized by shortened telomeres.

I. Methods of Extending Telomere Length in Cells of an Organism

This invention provides methods of extending telomere length in target cells of a subject. This is achieved by recombinant expression of active telomerase in the target cell. Telomerase is expressed recombinantly by the expression of an exogenously supplied nucleotide sequence encoding telomerase reverse transcriptase and/or telomerase RNA component. The nucleotide sequence is supplied by endocytosis of a targeting liposome that bears on its surface a targeting moiety that binds to a moiety on the surface of the target cell, and which contains in its compartment a polynucleotide vector having a nucleotide sequence encoding telomerase reverse transcriptase operatively linked with the expression control sequence that is active in the cell.

The subject can be any organism having telomeres, particularly those having cells that exhibit cellular senescence, for example as a result of lack of expression of active telomerase. This includes for example, vertebrates, mammals and humans.

Liposomes of this invention can be delivered to cells with shortened telomeres. For example, they can be delivered to cells and organisms that have conditions associated with shortened telomeres. These conditions include, without limitation, the diseases identified in Table 1, herein. In particular, this invention contemplates lengthening telomeres in cells of the central nervous system of individuals having Central Nervous System (CNS) disorders associated with shortened telomeres.

A variety of conditions associated with shortened telomeres can be alleviated by expression of telomerase in those cells with shortened telomeres. Such conditions include, without limitation, Alzheimer's disease, Parkinson's disease, arterial sclerosis, osteoporosis and progeria.

When the exogenous TERT gene is supplied on a vector figured for transient expression of telomerase reverse transcriptase in the cell, the risk of conditions associated with chronic over-expression of telomerase are decreased. Accordingly, the amount of telomerase can be metered, for example, by repeated administration of the liposome. Furthermore, the amount of telomerase can be regulated by the configuration of the expression construct. For example, the promoter on the construct can be selected for high level expression of the TERT nucleotide sequence. An expression construct for expressing telomerase RNA component also can increase expression. Increased expression results in increased lengthening of telomeres in the cell. Delivery of an expression construct that recombinantly expresses telomerase in a cell results in more sustained and effective expression of active telomerase in lengthening of telomeres than other methods of inducing expression of telomerase in a cell. Active telomerase functions to extend the length of telomeres in the cell. The methods of this invention produce levels of telomerase activity in cells of organisms at levels effective to produce measurable increases in telomere length.

The methods of this invention can result in expression of telomerase transiently (e.g., expression for up to any of 4 hours, 8 hours, 24 hours, 2 days, 4 days, or 8 days). Telomerase can be expressed in amounts of between 100 copies and 100,000 copies of telomerase in the target cell.

A therapeutically effective amount of the liposome will vary depending upon the individual being treated and the particular gene being administered. The appropriate dose will be established by procedures well known to those of ordinary skill in the art. The therapeutic treatment can be repeated at intervals of a few hours to intervals of a few weeks, with an interval of, e.g., 48 hours, to further extend cell telomeres or to target cells or tissues that were not effectively targeted by previous treatments. For some repeated treatments, the targeting agents used and the set of therapeutic genes used can vary according to the receptors endogenous to the tissues and the cells targeted.

The plasmids are expressed in subject cells natively and transiently, for a period of hours to days, with peak expression of the plasmid genes occurring approximately 48 hours after administration, resulting in the formation of multiple competent copies of the enzyme telomerase at cellular concentrations sufficient to extend chromosomal telomere structures of senescent or senescing cells by hundreds to thousands of base pairs, phenotypically rejuvenating subject cells, tissues and organisms—improving replicative and regenerative capacity, improving function and reducing stresses due to the reduction in cascading effects of senescent function; thereby treating and preventing age-related disorders and telomere-related disorders. Elevated expression of telomerase within subject cells can be transient only, so that after treatment, normal shortening of telomeres and cellular aging occurs with subsequent cell divisions.

In accordance with the current invention, the therapeutic treatment is repeated periodically, at intervals from several months to several years to restore telomere length lost since the previous treatment, or to further extend telomeres.

Targeting liposomes of this invention can be cell specific or can bind with cells systemically. Accordingly, cell specific expression can be achieved by using a promoter inducible in the target cell, but not in other cells.

One target is somatic stem cells (also referred to as adult stem cells) and progenitor cells. These cells can be targeted systemically or in a particular tissue. In adult organisms, stem cells and progenitor cells act as a repair system for the body, replenishing adult tissues.

Liposomes are administered by any route effective to deliver the liposome to the target cell. Liposomes can be delivered directly to the environment of the target cell. However, because they can be configured to cross epithelial barriers, the liposomes of this invention can be delivered to more easily accessed spaces, such as the blood stream. Target cells can be cells belonging, without limitation, to any of the cardiovascular system; the digestive system; the endocrine system; the urinary system; the immune system; the musculoskeletal system; the nervous system; the reproductive system; or the respiratory system.

Accordingly, the liposomes of this invention, combined with a suitable pharmaceutical carrier, can be provided through oral, transdermal, mucosal (e.g., nasal, sublingual, vaginal, buccal, or rectal) or parenteral (e.g., subcutaneous, intravenous, intra-arterial, intramuscular, intra-particular, intra-peritoneal, intraocular, intra-thecal injection) administration.

If the target cell is located within the compartment of delivery, then a targeting agent against the receptor on the target cell suffices. For example, cells can be reached by oral, transdermal, mucosal, intra-articular or intraocular administration. For example, if the target cell is a chondrocyte and liposome is delivered into the intra-articular space then, the liposome includes a targeting agent against a receptor on a chondrocyte.

The ability of the liposomes of this invention to cross an endothelial barrier allows access to cells not otherwise accessible through intravenous administration. If the target cell is in an organ system, such of the nervous system, for which direct access by injection is difficult, the liposome can be delivered intravenously.

A liposome of this invention can include one or more nucleic acid molecules having functional elements sufficient for delivery of a TERT-encoding molecule into the cell nucleus and its transcription there. For example, a nucleic acid molecule included in a liposome of this invention can comprise an expression cassette that comprises an expression control sequence that is operative in the target cell and that is operatively linked with a nucleotide sequence encoding TERT. Similarly, the same or different nucleic acid molecule can comprise an expression cassette comprising an expression control sequence that is operative in the target cell and that is operatively linked with a nucleotide sequence for TERC. This suffices for expression of the nucleic acid molecule once transported to the cell nucleus.

II. Targetable Liposomes

Liposomes of this invention have a phospholipid membrane having a surface and defining an internal compartment. The surface can be PEG-ylated. PEG-ylation extends the life of the liposome in an organism and is useful as a coupling moiety to couple with a targeting agent. The internal compartment comprises at least one nucleic acid vector having an expression control sequence operatively linked with a nucleotide sequence encoding telomerase reverse transcriptase. The vector can be a plasmid, for example, one that is not incorporated into chromosomal DNA and that results in transient expression of the telomerase reverse transcriptase sequences contained on it. The expression control sequence is selected to be active in the target cell. For example, it can include a constitutive promoter or promoter active specifically in the target cell. Other expressible gene constructs can be included in the compartment. They can be included on the same vector as the one encoding telomerase reverse transcriptase or on separate vectors. Other genes can be under the transcriptional control of the same promoter that controls expression of telomerase reverse transcriptase or a separate promoter.

A. Liposome Construction

One version of a targetable liposome is described in U.S. Pat. No. 6,372,250 (Pardridge). The liposomes can have diameters of less than 200 nanometers, e.g., diameters of between 50 and 150 nanometers or about 100 nanometers. Suitable types of liposomes can be made with neutral phospholipids such as 1-palmitoyl-2-oleoyl-sn-glycerol-3-phosphocholine (POPC), diphosphatidy phosphocholine, distearoylphosphatidylethanolamine (DSPE), or cholesterol, along with a small amount (1%) of cationic lipid, such as didodecyldimethylammonium bromide (DDAB) to stabilize the anionic DNA within the liposome.

The liposome can be constructed with polyethylene glycol-(PEG-)conjugated lipids, and the PEG strands on the surface of the liposome stabilizes the liposome in vivo and increases the plasma residence time. See, e.g., U.S. Pat. No. 6,132,763 (Fisher). Some fraction of the PEG molecules, for example, 1-2%, can carry a terminal maleimide functional group to allow for conjugation of the liposome surface with thiolated targeting agents.

Polyethylene glycol (PEG) is useful as a coupling moiety. Other coupling moieties, such sphingomyelin, can be used as well. The molecular weight of the coupling moiety can be between 1000 and 50,000 Da. Coupling moieties with a molecular weight of about 2000 Da, for example PEG with a molecular weight of about 2000 Da, also are useful. In one embodiment, a targeting agent is attached through reaction with a maleimide group attached to PEG-ylated lipid. Liposomes can have a plurality of targeting molecules attached thereto, for example, 5 to 1000 or 25-40.

Liposomes can be made by dispersing lipids, including PEG-ylated lipids and nucleic acid vectors, in 0.05 M Tris-Cl at pH 8.0 and sonicating for 10 minutes. The therapeutic genes can be encapsulated within the liposome according to any of the well-known drug encapsulation processes. For example, encapsulation by sonication, freeze/thaw, evaporation, the Mozafari method and extrusion through membrane filters. (Colas, J C; Shi, W; Rao, V S; Omri, A; Mozafari, M R; Singh, H (2007). “Microscopical investigations of nisin-loaded nanoliposomes prepared by Mozafari method and their bacterial targeting”. Micron (Oxford, England: 1993) 38 (8): 841-7.) Useful methods of liposome encapsulation include methods that provide a higher yield of unilamellar liposomes of the desired diameter, containing a higher percentage of vectors that are undamaged by the method of manufacture.

B. Targeting Molecules

Liposomes have on their surface a targeting agent directed against a cell moiety on a target cell, which moiety can mediate translocation of the liposome into the target cell by methods such as endocytosis or macropinocytosis. In the case in which the liposome is to be administered by a route that requires crossing of an endothelial barrier or other cellular membrane barriers, such as astrocytes and pericytes of the blood brain barrier, for access to the target cell, the liposome surface also can include a targeting agent directed against a moiety on an endothelial cell or other membrane barrier cell, which moiety mediates transcytosis across the barrier.

Endothelial barriers and other cellular membrane barriers in an organism include the endothelial lining of the cardiovascular system, including capillary walls, the blood brain barrier, the blood-ocular barrier, the blood retinal barrier, the blood-testes barrier, and the blood-ovaries barrier.

For the case of systemic or targeted delivery by way of the blood circulatory system, receptor molecules can be those that i) are prevalent in occurrence for the targeted cells; ii) effect both transcytosis of the liposome across membrane barriers and endocytosis of the liposome into the target cell; iii) effect the formation of a nuclear signaling endosome (NSE). (Charles L Howe, “Modeling the signaling endosome hypothesis: Why a drive to the nucleus is better than a (random) walk”, Theoretical Biology and Medical Modelling 2005, 2:43.)

For the case of targeted delivery of the therapy directly to cells tissues for which there is no need to cross a membrane barrier, such as the case of injection into the cartilage of a joint targeting chondrocytes, the receptor molecule does not require the ability to effect transcytosis across membrane barriers.

Examples of receptor molecules useful in the compositions and methods described herein are the insulin receptor, the insulin-like growth factor receptor, the epidermal growth factor receptor, and receptors for other hormones that are distributed through the blood circulatory system.

In certain embodiments, receptor molecules that result in the formation of endosomes that are destined for cellular destinations other than the nucleus, for example, a lysosome, are not used. Examples of such receptor molecules are the low-density lipid receptor or the transferrin receptor, which effect endosomes that are destined for the lysosome.

Targeting agents can be selected from, without limitation, an endogenous peptide ligand of the receptor, an analog of the endogenous peptide ligand, or a monoclonal antibody that binds the receptor. Endogenous ligands include, for example, insulin, insulin-like growth factor, epidermal growth factor, and other hormones. Useful ligands for the purposes of this invention include the insulin peptide or an analog of the same, such as the human insulin receptor monoclonal antibody (HIRMAb), as described by Boado and Pardridge. (Ruben J. Boado and William M. Pardridge, “The Trojan Horse Liposome Technology for Nonviral Gene Transfer across the Blood-Brain Barrier”, Journal of Drug Delivery, Volume 2011, Article ID 296151.)

Such agents can be coupled by thiolation and bonding to, for example, a maleimide or hydrizide moiety on PEG. Alternatively, a targeting agent can be conjugated via a disulfide linker to the liposome that has been reacted with N-succinimidyl 3-(2-pyridylthio) proprionate (SPDP). Alternatively, the targeting agent can be bound through an avidin-biotin interaction in which one molecule is coupled to avidin, and the other to biotin.

Monoclonal antibodies are useful as targeting agents. One such antibody is an antibody directed against human insulin receptor (HIRMAb). One such antibody has been asserted to deliver liposomes to the brain and enhance translocation of the plasmids across the nuclear membrane to the nuclear compartment. (R. J. Boado, “Blood-brain barrier transport of non-viral gene and RNAi therapeutics,” Pharmaceutical Research, vol. 24, no. 9, pp. 1772-1787, 2007.)

“Antibody” refers to a composition comprising a protein that binds specifically to a corresponding antigen and has a common, general structure of immunoglobulins. The term antibody specifically covers polyclonal antibodies, monoclonal antibodies, dimers, multimers, multispecific antibodies (e.g., bispecific antibodies), and antibody fragments, as long as they exhibit the desired biological activity. Antibodies can be humanized or chimeric. As used herein, antibody also includes an antigen binding portion of an immunoglobulin that retains the ability to bind antigen. These include, as examples, F(ab), a monovalent fragment of VL CL and VH CH antibody domains; and F(ab)2 fragment, a bivalent fragment comprising two Fab fragments linked by a disulfide bridge at the hinge region. The term antibody also refers to recombinant single chain Fv fragments (scFv) and bispecific molecules such as, e.g., diabodies, triabodies, and tetrabodies (see, e.g., U.S. Pat. No. 5,844,094).

Alternatively, the surface of the liposome could be conjugated with two different “transportable peptides,” one peptide targeting an endogenous capillary wall receptor or BBB receptor and the other targeting an endogenous cell membrane peptide. The latter could be specific for particular cells within the organism, such as neurons, glial cells, pericytes, smooth muscle cells, microglia, fibroblasts, myocytes, chondrocytes, osteoblasts, and other cells that may play important roles in human pathology.

C. Vector

Nucleic acid molecules in the liposomes of this invention typically are carried on a vector. The vector can be, for example, a plasmid, a synthetic chromosome or a virus, for example, the lentiviral vector. Plasmid vectors useful in this invention are available from, for example, Aldevron (Madison, Wis.). In certain embodiments, the vector is selected to have transient presence or activity in the cell. For example, the vector can be one that does not integrate into the chromosome of the cell. Plasmid vectors are useful for such purposes.

D. Expression Cassettes

Nucleotide sequences for expression cells typically are comprised in an expression cassette. Expression cassette includes an expression control sequence operative in the target cell and operatively linked with the nucleotide sequence to be expressed.

1. Expression Control Sequences

The expression control sequence can include any elements useful for promoting transcription and/or translation. Expression cassettes typically include a promoter. The promoter can be a constitutive promoter or an inducible promoter active in the target cell. Examples of useful constitutive promoters include the Simian vacuolating virus 40 (SV40) promoter, the cytomegalovirus (CMV) promoter and the respiratory syncytial virus (RSV) promoter. Inducible promoters useful in specific target cells include the smooth muscle gamma actin (SMGA) promoter for selective expression in smooth muscle cells; the human collagen type I a2 (hCol1a2) promoter for selective expression in osteoblasts; the flk-1 promoter for selective expression in endothelial cells; and the surfactant protein C promoter (SP-C) for selective expression in type-II pneumocytes.

A further method of effecting tissue specific expression control is by the incorporation of a DNA Nuclear Targeting Sequence (DTS) that combines with endogenous tissue specific transcription factors to mediate nuclear entry of plasmid DNA in a cell-specific manner, such as described in Miller. (Miller A M, Dean D A, “Tissue-specific and transcription factor-mediated nuclear entry of DNA”, Adv Drug Deliv Rev. 2009 Jul. 2:61(7-8):603-13.) As described in Miller at pp 607-608, examples of tissue specific DTS sequences can include a DTS derived from the smooth muscle gamma actin (SMGA) promoter, for nuclear import of plasmid DNA exclusively in smooth muscle cells; a DTS derived from the human collagen type I a2 (hCol1a2) promoter for nuclear import of plasmid DNA exclusively in osteoblasts; a DTS derived from the flk-1 promoter for nuclear import of plasmid DNA exclusively in endothelial cells; and a DTS derived from the surfactant protein C promoter (SP-C) for nuclear import of plasmid DNA exclusively in type-II pneumocytes.

A method of systemic or non-targeted expression control is by the incorporation a DTS that combines with constitutive endogenous transcription factors. As described in Miller et al. (Miller et al., Advanced Drug Delivery Reviews 61:603 (2009)) at pp 607-609, as little as 72 by of the SV40 enhancer is capable of driving plasmid nuclear import. (D. A. Dean, B. S. Dean, S. Muller, L. C. Smith, Sequence requirements for plasmid nuclear entry, Exp. Cell Res. 253 (1999) 713-722.)

2. Expressible Sequences

Therapeutic genes, which are encapsulated within the liposome, comprise at least one Telomerase Reverse Transcriptase (TERT) nucleotide sequence; and optionally at least one Telomerase RNA Component (TERC) nucleotide sequence; and optionally one or more copies of nucleotide sequences for the expression of cofactors for the extension of telomeres. Exemplary cofactor genes include LMNA, useful for the condition of Hutchinson-Gilford progeria syndrome or other short telomere conditions resulting from a cellular deficiency of competent Lamin A/C proteins; and Elongation Factor 1 (ELF1) for increased rates of cellular production of proteins required for telomere extension. The coding sequence also includes a polyadenylation sequence (pA) for the production of mature messenger RNA (mRNA) for translation.

Sequences for polynucleotides and for polypeptides also can be found at the NCBI website, www.ncbi.nlm.nih.gov/gene.

The therapeutic gene can have the naturally occurring sequence or can be a variation of the naturally occurring sequence that enhances processivity, or enhances the overall rate of telomere extension or enhances the overall extent of telomere extension.

In addition to the therapeutic genes, the vector DNA can also contain nucleotide sequences either before or after the therapeutic sequence and these additional parts of the plasmid can promote tissue-specific transcription of the plasmid in a particular cell, can promote enhanced translation and/or stabilization of the mRNA of the therapeutic gene, and can enable episomal replication of the transgene in cells.

The plasmids can all comprise identical therapeutic genes or can comprise different sets of the therapeutic genes. The plasmids contained in the liposomes can all be comprised of identical sets of therapeutic genes, or some of the liposomes can contain plasmids comprised of different sets of therapeutic genes. For example, one half of the liposomes can contain plasmids comprising the TERT gene, and one half of the liposomes can contain plasmids comprising the TERC gene and the LAMINA gene.

The number of different therapeutic genes encapsulated within the liposome can vary from 1 to many, depending on the disease being treated. The limiting factor will be the diameter of therapeutic gene that is encapsulated within the liposome. Using polycationic proteins such as histone, protamine, or polylysine, it is possible to compact the size of plasmid DNA that contains several thousand nucleotides to a structure that has a diameter of 10-30 nm. The volume of a 100 diameter liposome is 1000-fold and 35-fold greater than the volume of a 10 nm and 30 nm DNA compacted sphere, respectively. Therefore, it is possible to encapsulate many copies of the same gene or multiple copies of multiple genes within the liposome.

i. Telomerase Reverse Transcriptase

The telomerase reverse transcriptase can be any TERT that functions to extend telomeres in the target cell. Typically, the TERT will be a naturally occurring molecule from the genome of the species of the target cell. For example if the target cell is a human cell, the TERT can be human TERT. Alternatively, the TERT can be a modified form of TERT. The nucleotide sequence of a cDNA encoding human TERT is provided as SEQ ID NO:1. The amino acid sequence of human TERT is provided as SEQ ID NO:2. TERT sequences also can be found at the NCBI website as Gene ID: 7015. The full genomic sequence is given as 41898 nucleotides.

Alternatively, TERT can be a modified form of TERT having enhanced activity. Versions of such TERT variants or described in, e.g., U.S. Pat. No. 6,337,200 (Morin).

ii. Telomerase RNA Component

TERC is normally expressed in cells at low levels. However, in certain embodiments of the invention a nucleic acid molecule encoding TERC is co-delivered to the cell. There, it is expressed and associates with TERT, producing functional telomerase. An expression cassette to produce TERC can include an expression control sequence operatively linked with a nucleotide sequence encoding telomerase RNA component. A genomic nucleotide sequence for human telomerase RNA component is presented as SEQ ID NO:3. A nucleotide sequence for human telomerase RNA component is presented as SEQ ID NO:4. TERC sequences also can be found at the NCBI website as Gene ID: 7012.

TERC can be co-delivered with TERT either on the same vector or on a different vector. On the same vector, TERC can be under transcriptional control of the same or different promoter that controls expression of TERT.

iii. Lamin A

Lamin A/C, also known as LMNA, is a protein that in humans is encoded by the LMNA gene. Lamin A/C belongs to the lamin family of proteins. A truncated version of lamin A, known as progerin, is associated with Hutchinson-Gilford progeria syndrome. Accordingly, in embodiments in which the liposome of this invention is delivered to a subject having Hutchinson-Gilford progeria syndrome, the liposome also includes a vector containing an expression control sequence operative in the target cell and operatively linked to a nucleotide sequence encoding Lamin A/C. Again, a nucleotide sequence encoding Lamin A/C can be present on the same or different nucleic acid vector as any other sequence being delivered by the liposome, and under transcriptional control the same or different promoter. Homo sapiens lamin NC (LMNA) sequences can be found at the NCBI website as NCBI Reference Sequence: NG_008692.2.

iv. Other Genes

Liposomes of this invention also can include other expression cassettes for expressing other therapeutic nucleotide sequences, including those useful in enhancing activity and processivity of telomerase. Examples of such proteins include Elongation Factor 1 (ELF1), telomere repeat-binding factor 1 (TRF1), telomere repeat-binding factor 2 (TRF2), protection of telomeres protein 1 (POT1), and adrenocortical dysplasia protein (ACD or TPP1).

III. Pharmaceutical Compositions

This invention further provides pharmaceutical compositions comprising a liposome of this invention and a physiologically (i.e., pharmaceutically) acceptable carrier. The term “carrier” refers to a typically inert substance used as a diluent or vehicle for a diagnostic or therapeutic agent. The term also encompasses a typically inert substance that imparts cohesive qualities to the composition. Physiologically acceptable carriers can be liquid, e.g., physiological saline, phosphate buffer, normal buffered saline (135-150 mM NaCl), water, buffered water, 0.4% saline, 0.3% glycine, glycoproteins to provide enhanced stability (e.g., albumin, lipoprotein, globulin, etc.), and the like. Since physiologically acceptable carriers are determined in part by the particular composition being administered as well as by the particular method used to administer the composition, there are a wide variety of suitable formulations of pharmaceutical compositions of the present invention (See, e.g., Remington's Pharmaceutical Sciences, 17th ed., 1989.)

The compositions of the present invention can be sterilized by conventional, well-known sterilization techniques or can be produced under sterile conditions. Aqueous solutions can be packaged for use or filtered under aseptic conditions. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate.

Dosage forms can be prepared for mucosal (e.g., nasal, sublingual, vaginal, buccal, or rectal), parenteral (e.g., subcutaneous, intravenous, intramuscular, or intra-arterial injection, either bolus or infusion), oral, or transdermal administration to a subject. Examples of dosage forms include, but are not limited to: dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a subject, including suspensions (e.g., aqueous or non-aqueous liquid suspensions, oil-in-water emulsions, or a water-in-oil liquid emulsions), solutions, and elixirs; liquid dosage forms suitable for parenteral administration to a subject; and sterile solids (e.g., crystalline or amorphous solids) that can be reconstituted to provide liquid dosage forms suitable for parenteral administration to a subject.

Injectable (e.g., intravenous) compositions can comprise a solution of the liposome suspended in an acceptable carrier, such as an aqueous carrier. Any of a variety of aqueous carriers can be used, e.g., water, buffered water, 0.4% saline, 0.9% isotonic saline, 0.3% glycine, 5% dextrose, and the like, and can include glycoproteins for enhanced stability, such as albumin, lipoprotein, globulin, etc. Often, normal buffered saline (135-150 mM NaCl) will be used. The compositions can contain pharmaceutically acceptable auxiliary substances to approximate physiological conditions, such as pH adjusting and buffering agents, tonicity adjusting agents, wetting agents, e.g., sodium acetate, sodium lactate, sodium chloride, potassium chloride, calcium chloride, sorbitan monolaurate, triethanolamine oleate, etc. In some embodiments, the liposome composition can be formulated in a kit for intravenous administration.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The pharmaceutical preparation can be packaged or prepared in unit dosage form. In such form, the preparation is subdivided into unit doses containing appropriate quantities of the active component, e.g., according to the dose of the liposome. The unit dosage form can be a packaged preparation, the package containing discrete quantities of preparation, in unit-dose or multi-dose sealed containers, such as ampoules and vials. The composition can, if desired, also contain other compatible therapeutic agents.

The liposome can be administered by injection or infusion through any suitable route including but not limited to intravenous, subcutaneous, intramuscular or intraperitoneal routes. An example of administration of a pharmaceutical composition includes storing the liposome at 10 mg/ml in sterile isotonic aqueous saline solution for injection at 4° C., and diluting it in either 100 ml or 200 ml 0.9% sodium chloride for injection prior to administration to the subject. The liposome is administered by intravenous infusion over the course of 1 hour at a dose of between 0.2 and 10 mg/kg. In other embodiments, the liposome is administered by intravenous infusion over a period of between 15 minutes and 2 hours. In still other embodiments, the administration procedure is via sub-cutaneous bolus injection.

The dose of liposome is chosen in order to provide effective therapy for the subject and is in the range of about 0.001 mg/kg to about 1000 mg/kg. The dose can be repeated at an appropriate frequency.

Administration can be periodic, depending on the level and timing of expression of telomerase in the target cell. Depending on the route of administration, the dose can be administered, e.g., once every 1, 3, 5, 7, 10, 14, 21, or 28 days or longer (e.g., once every 2, 3, 4, or 6 months). In some cases, administration is more frequent, e.g., 2 or 3 times per day. The subject can be monitored to adjust the dosage and frequency of administration depending on therapeutic progress and any adverse side effects, as will be recognized by one of skill in the art.

Thus in some embodiments, additional administration is dependent on the progress of the subject, e.g., the subject is monitored between administrations.

Liposomes of this invention can be administered at the initial dosage of about 0.001 mg/kg to about 1000 mg/kg daily and adjusted over time. A daily dose range of about 0.01 mg/kg to about 500 mg/kg, or about 0.1 mg/kg to about 200 mg/kg, or about 1 mg/kg to about 100 mg/kg, about 5 to about 10 mg/kg, or about 10 mg/kg to about 50 mg/kg, can be used.

TABLE I
Diseases associated with shortened telomeres

```
Neurologic:
      Alzheimer's disease
      Parkinson's disease
      Age-related neurological changes (other than AD and PD, including loss of
      coordination, poor reflex function, loss of or decreased in sensation, etc.
      Age-related sleep dysfunctions
      Age-related auditory changes (including presbycusis, tinnitus, etc.)
      Age-related visual/ocular changes (including macular degeneration,
      presbyopia, cataracts, glaucoma, diabetes-related ocular disease, dry eye
      syndrome, loss of contrast perception, etc.)
Cardiovascular:
      Atherosclerosis
      Coronary artery disease (including myocardial infarction, sudden death, etc.)
      Carotid artery disease
      Stroke
      Hypertension
      Congestive heart failure
      Peripheral vascular disease
Pulmonary:
      Chronic obstructive pulmonary disease (COPD)
      Idiopathic pulmonary fibrosis
Gastrointestinal and oral:
      Dental changes (periodontal disease, gingivitis, etc.)
      Hepatic insufficiency (including changes in drug metabolism)
      Age-related gastrointestinal changes (including gastro-esophageal reflux
      disorder, etc.)
Endocrine:
      Diabetes type II (and insulin resistance)
      Endocrine senescence (including sex steroids, changes in vaginal mucosa,
      menopause, andropause, thyroid dysfunction, calcitonin changes, obesity,
      etc.)
Urogenital:
      Renal insufficiency
      Urogenital changes (including prostatic hypertrophy, erectile dysfunction, etc.)
Orthopedic/muscular:
      Age-related musculature changes (loss of muscle mass, loss of muscle
      strength, etc.)
      Osteoarthritis (including hips, knees, ankles, shoulders, elbows, wrists,
      cervical vertebrae, thoracic vertebrae, lumbar vertebrae, sacral vertebrae,
      articulations in the bones of the hand, articulations in the bones of the foot,
      temporo-mandibular joint)
      Osteoporosis (including increased risk of fractures, both traumatic and non-
      traumatic)
Hematologic, Immune system and cancer:
      Immune senescence (including chronic inflammation, rheumatoid arthritis,
      increased risk for pneumonia, sepsis, cellulitis, shingles, etc.)
      Dermal aging (including wrinkles, loss of elasticity, “liver spots”, delayed
      healing, thinning of the dermis and epidermis, loss of subcutaneous fat,
      decrease in sebum and oil production, etc.)
      Age-related cancers (and genomic instability)
      Age-related pancytopenia
      Bone marrow failure
Other telomere-related diseases:
      Dyskeratosis congenital (DKC)
      Liver failure secondary to DKC (including cryptogenic hepatic cirrhosis,
      noncirrhotic portal hypertension, etc.)
      Progeria (including Hutchinson-Gilford progeria, Werner's progeria)
      Acrogeria
      Metageria
      Hoyeraal-Hreidarsson syndrome
      Bone marrow failure
      Acquired aplastic anemia
      HIV (as AIDS secondary to telomere loss and secondary cell senescence)
```

All publications, patents, and patent applications mentioned in this specification are herein incorporated by reference to the same extent as if each individual publication, patent, or patent application was specifically and individually indicated to be incorporated by reference.

While preferred embodiments of the present invention have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of expressing telomerase in a target cell in a subject comprising administering to the subject a pharmaceutical composition comprising a liposome and a pharmaceutically acceptable carrier;
wherein the liposome comprises a PEG-ylated lipid membrane having an external surface and defining an internal compartment, wherein:
a) the external surface has attached thereto a targeting agent directed against a receptor on a target cell involved in receptor-mediated endocytosis or macropinocytosis; and
b) the internal compartment contains a nucleic acid vector comprising an expression control sequence operative in the target cell and operatively linked to a nucleotide sequence encoding telomerase reverse transcriptase (TERT);
wherein the liposome is endocytosed or macropenocytosed by the target cell, and the target cell expresses active telomerase.

2. The method of claim 1 wherein the internal compartment contains nucleic acid vector comprising an expression control sequence operative in the target cell and operatively linked to a nucleotide sequence encoding telomerase RNA component (TERC).

3. The method of claim 1 wherein the subject is a vertebrate, e.g., a mammal, e.g., a human.

4. The method of claim 1 wherein the pharmaceutical composition is administered intravenously, intrathecally, intra-articularly, intraocularly, intramuscularly, orally, parenterally or topically.

5. The method of claim 1 wherein the pharmaceutical composition is administered intravenously, and wherein the external surface of the liposome has attached thereto a targeting agent directed against a receptor on an endothelial cell of an endothelial barrier involved in receptor-mediated transcytosis, wherein the liposome crosses the endothelial barrier.

6. The method of claim 5 wherein the endothelial barrier is the blood brain barrier.

7. The method of claim 5 or 6 wherein the targeting agent directed against the receptor on the target cell is also directed against the receptor on the endothelial cell.

8. The method of claim 7 wherein the target cell is a cell of the nervous system and the endothelial cell is a cell of the blood brain barrier.

9. The method of claim 1 wherein the target cell is a cell of the cardiovascular system; the digestive system; the endocrine system; the urinary system; the immune system; the musculoskeletal system; the nervous system; the reproductive system; or the respiratory system.

10. The method of claim 1 wherein the liposome is administered intra-articularly and wherein the liposome comprises a targeting agent for a receptor on a chondrocyte.

11. The method of claim 1 wherein the subject suffers from a disease associated with shortened telomeres.

12. The method of claim 11 wherein the active telomerase extends the length of shortened telomeres in the subject.

13. A method of treating a subject suffering from a disease associated with shortened telomeres comprising administering to the subject a liposome,
wherein the liposome comprises a PEG-ylated lipid membrane having an external surface and defining an internal compartment, wherein:
a) the external surface has attached thereto a targeting agent directed against a receptor on a target cell involved in receptor-mediated endocytosis or macropinocytosis; and
b) the internal compartment contains a nucleic acid vector comprising an expression control sequence operative in the target cell and operatively linked to a nucleotide sequence encoding telomerase reverse transcriptase (TERT);
wherein administration results in expression of active telomerase in target cells of the subject and wherein the active telomerase extends the length of telomeres in the target cells.

14. The method of claim 13 wherein expression of telomerase is transient (e.g., expression for up to any of 4 hours, 8 hours, 24 hours, 2 days, 4 days, or 8 days).

15. The method of claim 13 wherein the administration produces between 100 copies and 100,000 copies of telomerase in the target cell.

16. The method of claim 13 wherein the disease associated with shortened telomeres is selected from Alzheimer's disease, arterial sclerosis, osteoporosis and progeria.

17. The method of claim 13 comprising administering a liposome to the subject a plurality of times.

18. A liposome for delivering at least one nucleic acid vector encoding telomerase reverse transcriptase to a target cell, wherein the liposome comprises a PEG-ylated lipid membrane having an external surface and defining an internal compartment, wherein:
a) the external surface has attached thereto a targeting agent directed against a receptor on a target cell involved in receptor-mediated endocytosis or macropinocytosis; and
b) the internal compartment contains a nucleic acid vector comprising an expression control sequence operative in the target cell and operatively linked to a nucleotide sequence encoding telomerase reverse transcriptase (TERT).

19. The liposome of claim 18 wherein the target cell is a cell of the nervous system, e.g., a neuron (e.g., a pyramidal cell, a Purkinje cell, a granule cell or another resident neuron), a glial cell (e.g., a microglial cell, an astroglial cell, an oligodendrocyte or another resident glial cell), or a transient cell entering the nervous system.

20. The liposome of claim 18 wherein the target cell is a cell of the cardiovascular system; the digestive system; the endocrine system; the urinary system; the immune system; the musculoskeletal system; the nervous system; the reproductive system; or the respiratory system.

21. The liposome of claim 18 wherein the targeting agent is directed toward an insulin receptor.

22. The liposome of claim 18 wherein the targeting agent is directed toward a target other than one that mediates formation of endosomes destined for cell destination other than the nucleus, e.g., other than transferrin receptor for low-density lipid receptor.

23. The liposome of claim 21 wherein the targeting agent comprises monoclonal antibody directed against the receptor.

24. The liposome of claim 18 wherein the targeting agent is selected from an endogenous peptide ligand of the receptor, an analog of the endogenous peptide ligand, or a monoclonal antibody that binds the receptor.

25. The liposome of claim 18 wherein the targeting agent is selected from insulin, insulin-like growth factor (IGF), and leptin.

26. The liposome of claim 18 wherein the external surface has attached thereto a targeting agent directed against a receptor on an endothelial cell of an endothelial barrier involved in receptor-mediated transcytosis.

27. The liposome of claim 19 wherein the endothelial barrier is the blood brain barrier.

28. The liposome of claim 27 wherein the targeting agent directed against a receptor on the target cell is also directed against the receptor on the endothelial cell.

29. The liposome of claim 28 wherein the target cell is a cell of the nervous system and the endothelial cell is a cell of the blood brain barrier.

30. The liposome of claim 18 wherein at least one nucleic acid vector comprises a plasmid.

31. The liposome of claim 18 wherein the TERT is a human TERT or a modified version thereof.

32. The liposome of claim 18 wherein the nucleotide sequence encoding TERT comprises a naturally occurring nucleotide sequence encoding human TERT.

33. The liposome of claim 18 wherein the expression control sequence comprises an SV40 promoter.

34. The liposome of claim 18 wherein at least one expression control sequence comprises a promoter specific for the target cell.

35. The liposome of claim 18 wherein the internal compartment further contains nucleic acid vector comprising an expression control sequence operative in the target cell and operatively linked to a nucleotide sequence encoding telomerase RNA component (TERC).

36. The liposome of claim 35 wherein TERC is human TERC.

37. The liposome of claim 35 wherein the nucleotide sequence encoding TERT and the nucleotide sequence encoding TERC are comprised in the same vector.

38. The liposome of claim 35 wherein the nucleotide sequence encoding TERT and the nucleotide sequence encoding TERC are operatively linked to the same expression control sequence.

39. The liposome of claim 18 wherein the internal compartment contains nucleic acid vector comprising an expression control sequence operative in the target cell and operatively linked to a nucleotide sequence encoding a therapeutic biomolecule other than telomerase reverse transcriptase or telomerase RNA component.

40. The liposome of claim 39 wherein the therapeutic biomolecule is LMNA A, e.g., human LMNA A.

41. The liposome of claim 39 wherein the therapeutic biomolecule is Elongation Factor 1 (ELF1).

42. A pharmaceutical composition comprising a liposome of any of claims 18 to 41 and a pharmaceutically acceptable carrier.

43. The pharmaceutical composition of claim 42 formulated for intravenous, intrathecal, intra-articular, intraocular, intramuscular, oral, parenteral or topical administration.

44. A unit dosage form comprising a container containing a pharmaceutical composition comprising a liposome of claim 18 this invention and a pharmaceutically acceptable carrier.


SEQUENCE LISTING (4 sequences)

SEQ ID NO: 1 - DNA, 4015 bp, Homo sapiens
gcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 2 - protein, 1069 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 3 - DNA, 965 bp, Homo sapiens
gggttgcggagggtgggcctgggaggggtggtggccattttttgtctaaccctaactgagaagggcgtaggcgccgtgcttttgctccccgcgcgctgtttttctcgctgactttcagcgggcggaaaagcctcggcctgccgccttccaccgttcattctagagcaaacaaaaaatgtcagctgctggcccgttcgcctcccggggacctgcggcgggtcgcctgcccagcccccgaaccccgcctggaggccgcggtcggcccggggcttctccggaggcacccactgccaccgcgaagagttgggctctgtcagccgcgggtctctcgggggcgagggcgaggttcaccgtttcaggccgcaggaagaggaacggagcgagtccccgcgcgcggcgcgattccctgagctgtgggacgtgcacccaggactcggctcacacatgcagttcgctttcctgttggtggggggaacgccgatcgtgcgcatccgtcacccctcgccggcagtgggggcttgtgaacccccaaacctgactgactgggccagtgtgctgcaaattggcaggagacgtgaaggcacctccaaagtcggccaaaatgaatgggcagtgagccggggttgcctggagccgttcctgcgtgggttctcccgtcttccgctttttgttgccttttatggttgtattacaacttagttcctgctctgcagattttgttgaggtttttgcttctcccaaggtagatctcgaccagtccctcaacggggtgtggggagaacagtcatttttttttgagagatcatttaacatttaatgaatatttaattagaagatctaaatgaacattggaaattgtgttcctttaatggtcatcggtttatgccagaggttagaagtttcttttttgaaaaattagaccttggcgatgaccttgagcagtaggatataacccccacaagctt

SEQ ID NO: 4 - RNA, 448 nt, Homo sapiens
ggguugcggagggugggccugggaggggugguggccauuuuuugucuaacccuaacugagaagggcguaggcgccgugcuuuugcuccccgcgcgcuguuuuucucgcugacuuucagcgggcggaaaagccucggccugccgccuuccaccguucauucuagagcaaacaaaaaaugucagcugcuggcccguucgccucccggggaccugcggcgggucgccugcccagcccccgaaccccgccuggaggccgcggucggcccggggcuucuccggaggcacccacugccaccgcgaagaguugggcucugucagccgcgggucucucgggggcgagggcgagguucaccguuucaggccgcaggaagaggaacggagcgaguccccgcgcgcggcgcgauucccugagcugugggacgugcacccaggacucggcucacacaugc